(19) United States
(12) Patent Application Publication
(10) Pub. No.: US 2026/0196327 A1
(43) Pub. Date: Jul. 9, 2026

(54) MACHINE VISION-BASED MEDICATION COMPLIANCE MONITORING SYSTEM AND RELATED TECHNIQUES

(71) Applicant: ProMed Innovations LLC, Alpharetta, GA (US)

(72) Inventors: Robert Page Ufford, Roswell, GA (US); Robert Kendall Ufford, Acworth, GA (US)

(21) Appl. No.: 19/443,118

(22) Filed: Jan. 8, 2026

Related U.S. Application Data
(60) Provisional application No. 63/743,270, filed on Jan. 9, 2025.

(51) Int. Cl. G16H 20/13; G16H 10/60; G16H 40/63; G16H 40/67; G16H 50/30

(52) U.S. Cl. CPC G16H 20/13; G16H 10/60; G16H 40/63; G16H 40/67; G16H 50/30

(57) ABSTRACT

A machine vision-based medication compliance monitoring system and related techniques are disclosed. In accordance with some embodiments, the disclosed system and techniques may involve detecting and verifying the retrieval of medication from medication packages such as blister packs. To such ends, the disclosed system and techniques may utilize an image capture device (e.g., a camera or video camera) integrated with one or more machine vision algorithms to identify when a user has interacted with the medication package (e.g., broken a blister pack cavity to access a medication dosage contained therein). As will be appreciated in light of this disclosure, the disclosed system and techniques may help to ensure accurate tracking of medication consumption events, enabling improved compliance monitoring and facilitating real-time or logged adherence data for healthcare providers, caregivers, and/or patients, in accordance with some embodiments.

### CROSS-REFERENCE TO RELATED APPLICATIONS

This patent application claims the benefit of U.S. Provisional Patent Application No. 63/743,270, titled “Machine Vision-Based Medication Compliance Monitoring System and Related Techniques,” filed on Jan. 9, 2025, which is herein incorporated by reference in its entirety.

### FIELD OF THE DISCLOSURE

The present disclosure relates to medication compliance monitoring and, more particularly, to machine vision-based medication compliance monitoring systems and techniques.

### BACKGROUND

Medications used in health care and treatment are often prescribed by medical professionals with instructions for the patient to take specific doses of the medication at specific times or intervals. To assist with proper dosage, the medications are often packaged in compartments of a blister package (i.e., a plastic material container comprising a number of sealable compartments having an open side intended to accommodate a drug dosage). These compartments are covered with a breakable cover. The blister pack is covered with a single breakable material, such as foil, or individual breakable flaps, in order to obtain a tight closure of the compartments and secure individual packaging. Generally, the compartments are arranged as a matrix configuration including a number of lines and/or columns. Typically, the rows can be times of the day, and the columns can be days of the week or month. Blister packs are commonly used to dispense oral solid medications, offering a degree of dose protection and organization.

### SUMMARY

The subject matter of this application may involve, in some cases, interrelated products, alternative solutions to a particular problem, and/or a plurality of different uses of a single system or article.

A first example embodiment provides a method of medication compliance monitoring utilizing a machine vision-based medication compliance monitoring system. The method includes performing, via the system, a first image capture of a medication package disposed within a designated image capture zone of the system. The method further includes retrieving, via the system, a medication schedule associated with a patient and pertaining to medication doses contained by the medication package. The method further includes outputting, via the system, at least one first signal indicative of at least one of: (1) a confirmation that the medication schedule is up to date; (2) an alert that a first designated time interval for the patient to dispense a medication dose from the medication package has begun; (3) an alert that the first designated time interval for the patient to dispense the medication dose from the medication package has expired; and (4) a current status of wired and/or wireless communication abilities of the system. The method further includes detecting, via the system, evidence of interaction of the patient with the medication package. The method further includes performing, via the system, one of the following after the medication dose has been dispensed from the medication package: (I) if (a) the medication package has been returned to the designated image capture zone within a second designated time interval, then (b) perform, via the system, a second image capture of the medication package and (c) update, via the system, a backend software regarding the same; or (II) if (a) the medication package has not been returned to the designated image capture zone within the second designated time interval, then (b) alert, via the system, at least one of the following regarding the same: (i) the backend software; (ii) the patient; and (iii) a third-party designated to monitor medication compliance of the patient.

In some cases, the detecting evidence of interaction of the patient with the medication package includes: detecting removal and return of the medication package from a field of view of the system utilizing at least one of machine vision and proximity sensing.

In some cases, in detecting evidence of interaction of the patient with the medication package, the system is configured to detect and analyze medication interaction both locally and remote to the system.

In some cases, the method further includes: learning, via the system, a plurality of forms of medication packages.

In some cases, the method further includes: applying, via the system, a date and a timestamp associated with a given target activity.

In some cases, the method further includes: detecting and recording, via the system, an undesired or excess dosage activity.

In some cases, the method further includes: retrieving, via the system, a schedule for obtaining at least one vital sign measurement associated with the patient; receiving, from a source external to the system, data pertaining to the at least one vital sign measurement associated with the patient; and sending, via the system, at least a portion of the data pertaining to the at least one vital sign measurement along with time and date information to a database external to the system. In some such instances, the method further includes outputting, via the system, at least one second signal indicative of at least one of: the at least one vital sign measurement; and an event in the schedule for obtaining the at least one vital sign measurement. In some other such instances, the at least one second signal is indicative of both: the at least one vital sign measurement; and an event in the schedule for obtaining the at least one vital sign measurement. In some other such instances, the method further includes analyzing, via the system, a relationship between (a) a medication compliance determination associated with the patient and (b) the at least one vital sign measurement.

A second example embodiment provides a machine vision-based medication compliance monitoring system configured to perform the method described above in relation to the first example embodiment.

In some cases, the system includes at least one image capture device configured to perform at least one of: the first image capture; and the second image capture. In some such instances, the at least one image capture device is a component native to a computing device. In some such instances, the computing device is a smartphone or a tablet computer. In some instances, the at least one image capture device is configured with a fixed field of view. In some other instances, the at least one image capture device is configured with an adjustable field of view.

In some cases, the system includes at least one indicator configured to output at least one of: the at least one first signal; and the at least one second signal.

In some cases, the system includes at least one display configured to provide a user interface or graphical user interface. In some such instances, the at least one display is touch-sensitive. In some other such instances, the at least one display is native to a computing device.

The features and advantages described herein are not all-inclusive and, in particular, many additional features and advantages will be apparent to one of ordinary skill in the art in view of the drawings, specification, and claims. Moreover, it should be noted that the language used in the specification has been selected principally for readability and instructional purposes and not to limit the scope of the inventive subject matter.

### BRIEF DESCRIPTION OF THE DRAWINGS

FIG. 1A illustrates a front elevation view of a machine vision-based medication compliance monitoring system configured in accordance with an embodiment of the present disclosure.

FIG. 1B illustrates a side elevation view of the system of FIG. 1A.

FIG. 2 illustrates an isometric view of a machine vision-based medication compliance monitoring system configured in accordance with an embodiment of the present disclosure.

FIG. 3 illustrates a front elevation view of the system of FIG. 2.

FIG. 4 illustrates another isometric view of the system of FIG. 2.

FIG. 5 illustrates a side elevation view of the system of FIG. 2.

FIG. 6 illustrates an isometric view of a machine vision-based medication compliance monitoring system configured in accordance with an embodiment of the present disclosure.

FIG. 7 illustrates another isometric view of the system of FIG. 6.

FIG. 8 illustrates an isometric view of a machine vision-based medication compliance monitoring system incorporating a computing device in accordance with another embodiment of the present disclosure.

FIG. 9 illustrates an isometric view of a machine vision-based medication compliance monitoring system incorporating a computing device in accordance with another embodiment of the present disclosure.

FIG. 10 is a flow diagram illustrating a method of medication compliance monitoring utilizing a machine vision-based medication compliance monitoring system in accordance with an embodiment of the present disclosure.

These and other features of the present embodiments will be understood better by reading the following detailed description, taken together with the figures herein described. In the drawings, each identical or nearly identical component that is illustrated in various figures may be represented by a like numeral. For purposes of clarity, not every component may be labeled in every drawing. Furthermore, as will be appreciated in light of this disclosure, the accompanying drawings are not intended to be drawn to scale or to limit the described embodiments to the specific configurations shown.

### DETAILED DESCRIPTION

A machine vision-based medication compliance monitoring system and related techniques are disclosed. In accordance with some embodiments, the disclosed system and techniques may involve detecting and verifying the retrieval of medication from medication packages such as blister packs. To such ends, the disclosed system and techniques may utilize an image capture device (e.g., a camera or video camera) integrated with one or more machine vision algorithms to identify when a user has interacted with the medication package (e.g., broken a blister pack cavity to access a medication dosage contained therein). As will be appreciated in light of this disclosure, the disclosed system and techniques may help to ensure accurate tracking of medication consumption events, enabling improved compliance monitoring and facilitating real-time or logged adherence data for healthcare providers, caregivers, and/or patients, in accordance with some embodiments. Numerous configurations and variations will be apparent in light of this disclosure.

### General Overview

Medication non-adherence is a significant challenge in healthcare, contributing to poor health outcomes, increased hospitalizations, and higher healthcare costs. Current blister packs do not inherently provide reliable confirmation of whether individual doses have been removed at the correct time. Existing medication adherence solutions include electronic pill bottles, smart blister packs with embedded sensors, and manual self-reporting. However, these approaches often require specialized hardware, increased manufacturing costs, or user behavior changes. As such, there remains a significant need for a low-cost, scalable, and device-agnostic solution that can determine dosage removal from standard blister packaging without requiring modification to the packaging itself.

Thus, and in accordance with some embodiments of the present disclosure, a machine vision-based medication compliance monitoring system and related techniques are disclosed. In accordance with some embodiments, the disclosed system and techniques may involve detecting and verifying the retrieval of medication from medication packages such as blister packs. To such ends, the disclosed system and techniques may utilize an image capture device (e.g., a camera or video camera) integrated with one or more machine vision algorithms to identify when a user has interacted with the medication package (e.g., broken a blister pack cavity to access a medication dosage contained therein). As will be appreciated in light of this disclosure, the disclosed system and techniques may help to ensure accurate tracking of medication consumption events, enabling improved compliance monitoring and facilitating real-time or logged adherence data for healthcare providers, caregivers, and/or patients, in accordance with some embodiments.

Some embodiments disclosed herein may realize one or more advantages and benefits as compared to existing approaches. For instance, in accordance with some embodiments, the disclosed system and techniques may be compatible for use in all light conditions. In accordance with some embodiments, the disclosed system and techniques may permit for detecting and learning new forms of medication packaging beyond those initially known (if any). In accordance with some embodiments, the disclosed system and techniques may provide for the ability to date and timestamp all activity accurately and a given desired frequency of occurrence, thus permitting accurate analysis of the relationship between the medication(s) and vital signs of the patient. In accordance with some embodiments, the disclosed system and techniques may provide for the ability to detect and analyze medication interaction both locally and remotely, thus helping to keep data transmission costs lower. In accordance with some embodiments, the disclosed system and techniques may provide for the ability to detect and record undesired or excess dosage activities. In accordance with some embodiments, the disclosed system and techniques may provide for the ability to detect (e.g., through vision, proximity sensing, or other suitable sensing capabilities) the removal and return of a medication package from the field of view. In accordance with some embodiments, the disclosed system and techniques may permit provision of performance feedback to the patient via visual, auditory, and/or electronic message formats.

As will be further appreciated in light of this disclosure, some embodiments of the disclosed system and techniques may exhibit a wide range of advantages over existing technologies, including any one or more of those listed in Table #1 below:

TABLE #1

May not require modification to standard blister packaging
May not require specialized hardware
May use ubiquitous mobile computing devices
May be scalable across medications and manufacturers
May support local, cloud, and/or hybrid artificial intelligence
(AI) deployment
May reduce cost and improve accessibility

### System Architecture and Operation

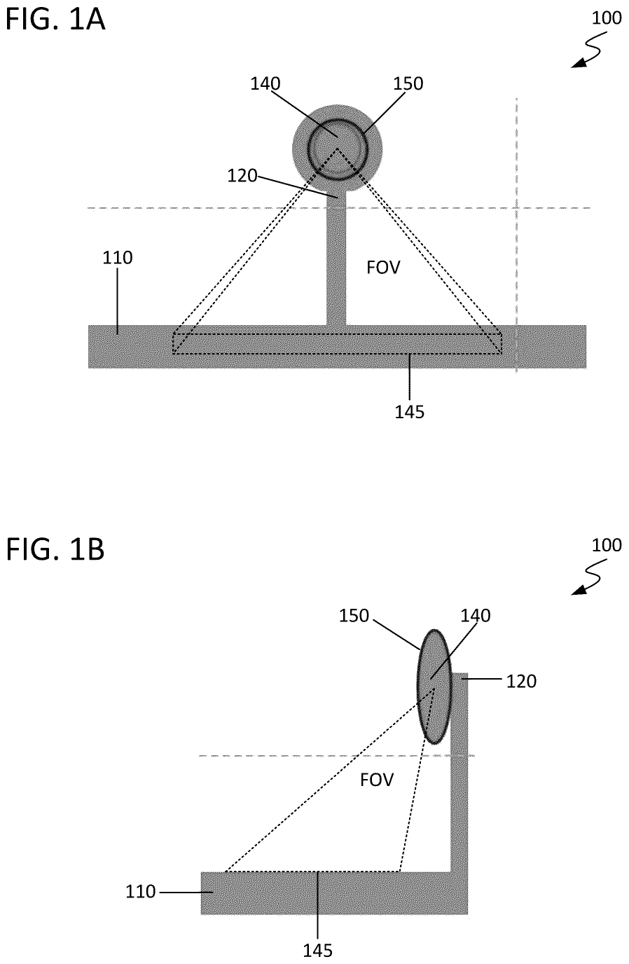

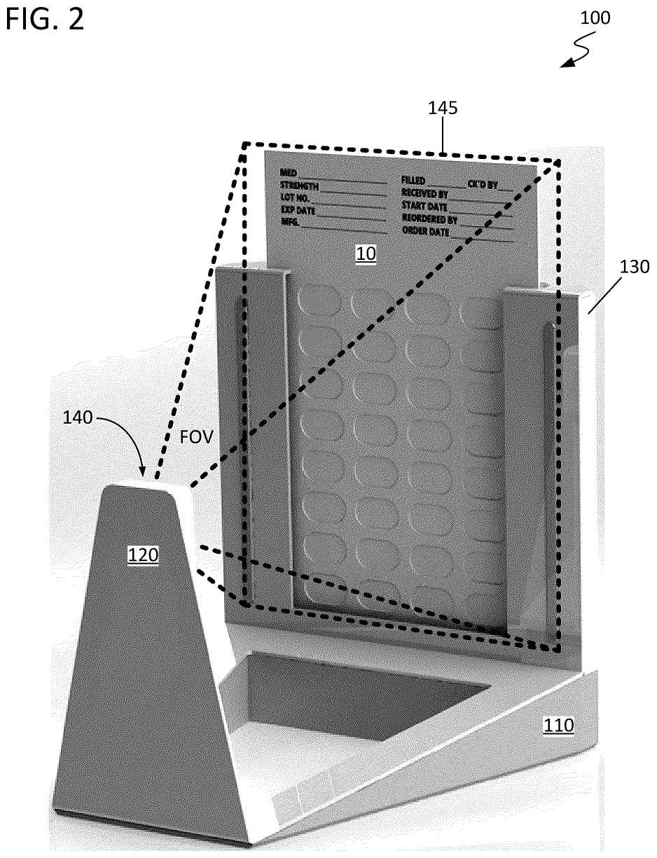

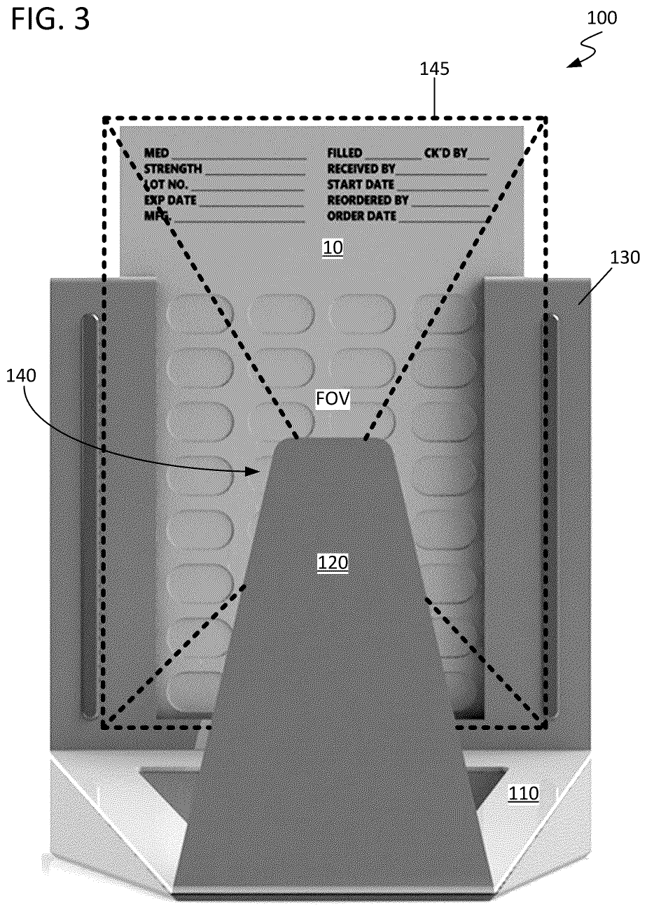

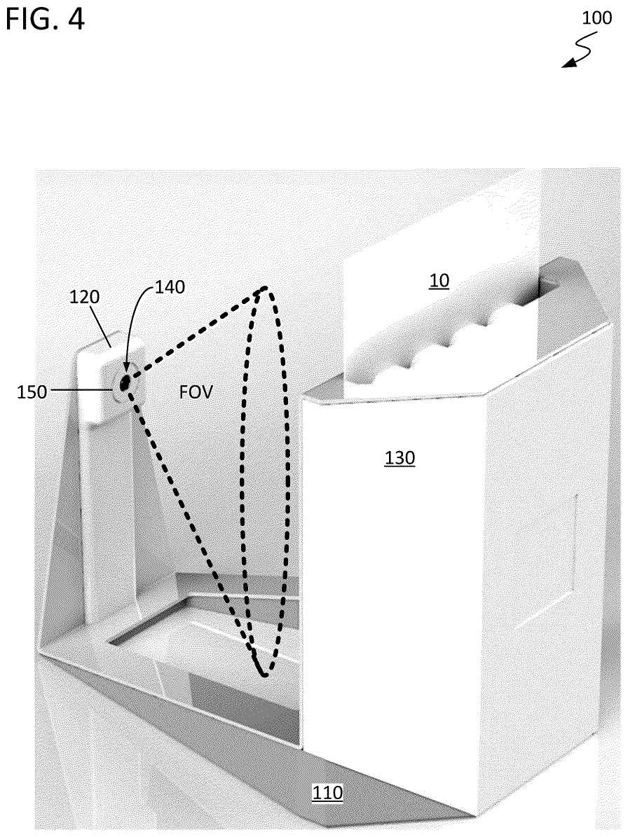

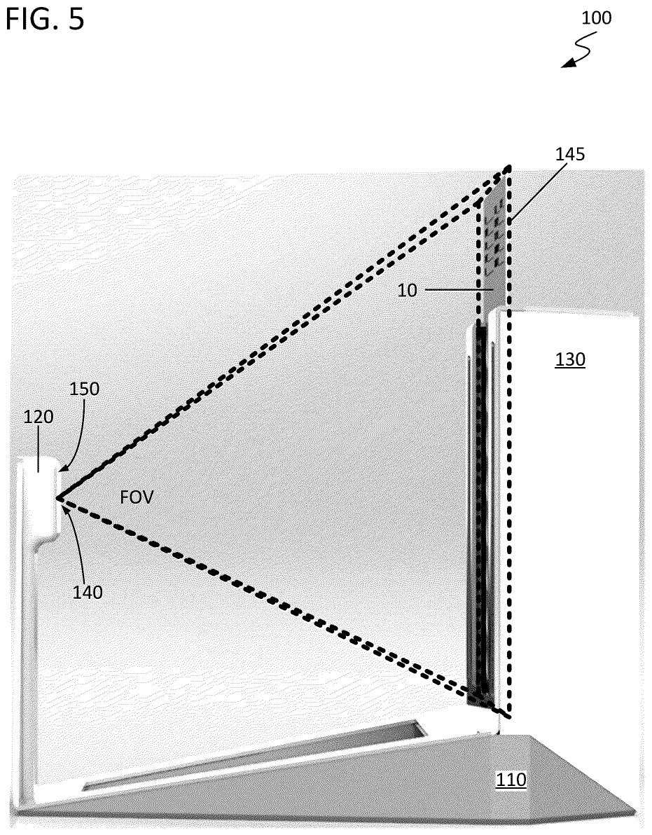

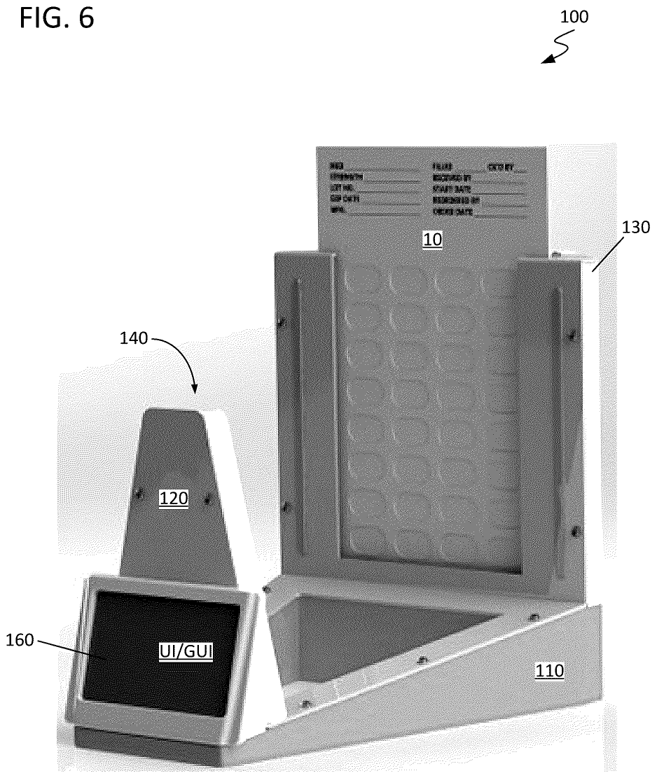

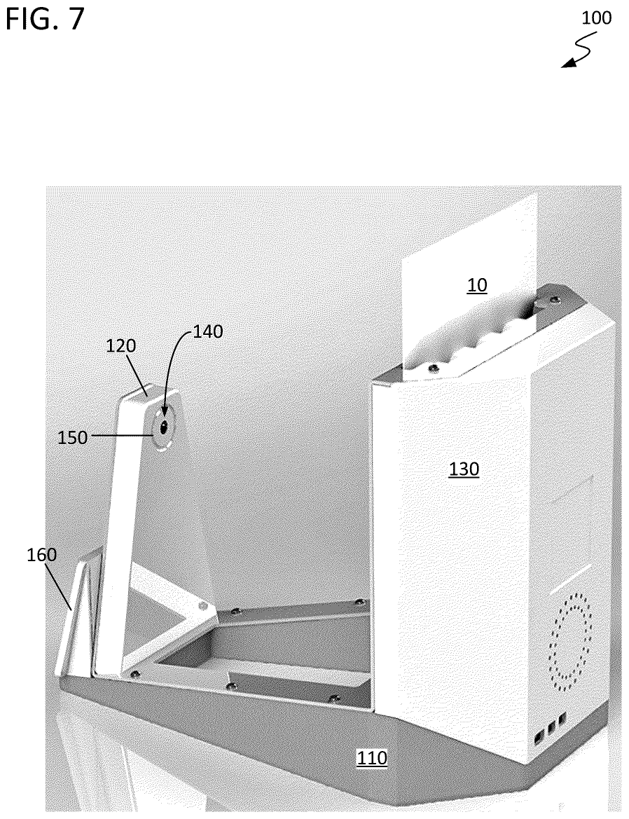

FIGS. 1A-1B illustrate several views of a machine vision-based medication compliance monitoring system 100 configured in accordance with an embodiment of the present disclosure. FIGS. 2-5 illustrate several views of a machine vision-based medication compliance monitoring system 100 configured in accordance with another embodiment of the present disclosure. FIGS. 6-7 illustrate several views of a machine vision-based medication compliance monitoring system 100 configured in accordance with another embodiment of the present disclosure.

As can be seen from the figures, system 100 may include a base portion 110, which generally may be configured to provide structural support for one or more other elements of system 100 against a given surface on which system 100 may be deployed. To that end, the specific construction of base portion 110 may be customized, as desired for a given target application or end-use. In some cases, base portion 110 may house one or more electronics utilized in operation of system 100.

In accordance with some embodiments, system 100 may include an image capture device mounting portion 120, at which an image capture device 140 (discussed below) may be mounted or installed, in temporary or permanent manner. The dimensions and geometry of image capture device mounting portion 120 may be customized, as desired for a given target application or end-use. In some cases, image capture device mounting portion 120 and base portion 110 may be of monolithic construction (i.e., single-piece construction), whereas in other cases, polylithic construction (i.e., multi-piece construction) may be provided, as desired. In some embodiments, system 100 may be configured such that image capture device 140 is structurally integrated, in part or in whole, with image capture device mounting portion 120. In this manner, image capture device mounting portion 120 may serve to house image capture device 140, in part or in whole. Other suitable configurations and capabilities for image capture device mounting portion 120 will depend on a given target application or end-use and will be apparent in light of this disclosure.

In accordance with some embodiments, system 100 may be configured simply to have a given medication package 10 (discussed below) laid before image capture device 140 within an image capture zone 145 (e.g., on base portion 110) to effectuate image capture thereof. In accordance with some other embodiments, though, system 100 optionally may include a designated medication package receiving portion 130, at which medication package 10 may be held or otherwise positioned, in temporary or permanent manner. The dimensions and geometry of medication package receiving portion 130 may be customized, as desired for a given target application or end-use. In some cases, medication package receiving portion 130 and base portion 110 may be of monolithic construction (i.e., single-piece construction), whereas in other cases, polylithic construction (i.e., multi-piece construction) may be provided, as desired.

Generally, medication package receiving portion 130 may be configured to receive and retain medication package 10, in part or in whole. In some embodiments, medication package receiving portion 130 may be configured to have medication package 10 at least partially inserted therein (e.g., from a top or side entry position). In some embodiments, medication package receiving portion 130 may be configured to have medication package 10 leaned (e.g., on edge) up against a supportive backing. In some embodiments, medication package receiving portion 130 may include one or more physical retention features (e.g., clips, clamps, fasteners, biasing elements, bezels, etc.) configured to physically retain medication package 10 thereat. In some embodiments, medication package receiving portion 130 may include one or more adhesive features for adhesion to medication package 10. In some embodiments, medication package receiving portion 130 may include one or more magnetic features for magnetic connection to medication package 10. Other suitable configurations and capabilities for medication package receiving portion 130 will depend on a given target application or end-use and will be apparent in light of this disclosure.

As can be seen further from the figures, system 100 also may include an image capture device 140, which may be (or otherwise may include) a camera and/or a video camera of a given target configuration and having one or more suitable image sensors. In accordance with some embodiments, image capture device 140 may be configured to capture image(s) of a medication package 10 (and/or other objects of interest) within a designated image capture zone 145 of system 100. The field of view (FOV) of image capture device 140 may be oriented to cover, in part or in whole, the designated image capture zone 145. Image capture device 140 may have a fixed or adjustable field of view, as desired for a given target application or end-use. Additionally, the image capture rate of image capture device 140 may be customized, as desired for a given target application or end-use. Storage of any image(s) captured by image capture device 140 may be performed locally (e.g., in memory of image capture device 140 itself) and/or remotely (e.g., in memory of a server database external to image capture device 140 and, in some cases, external to system 100).

In some cases, image capture device 140 may be fixedly mounted or installed at image capture device mounting portion 120, such that image capture device 140 is not adjustable in its physical orientation. In other cases, however, adjustable mounting or installation may be provided, such that the physical orientation of image capture device 140 may be adjusted in one or more directions (e.g., roll, pitch, and/or yaw), as desired. Other suitable configurations and capabilities for image capture device 140 will depend on a given target application or end-use and will be apparent in light of this disclosure.

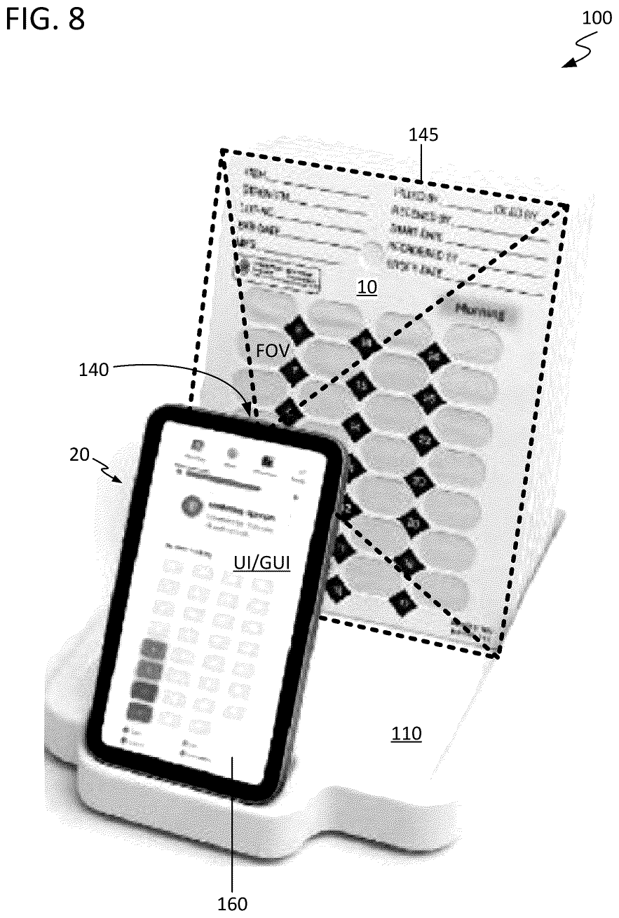

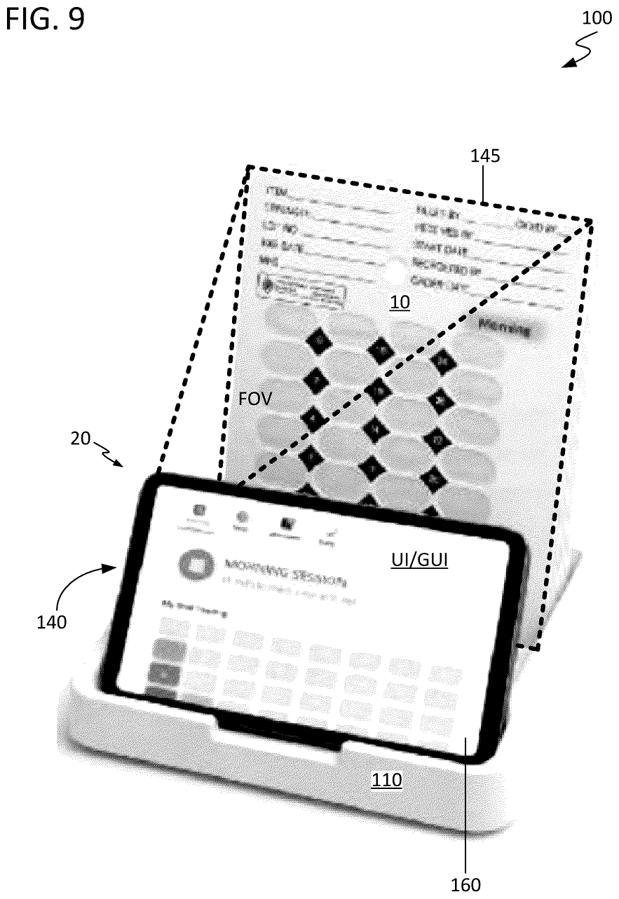

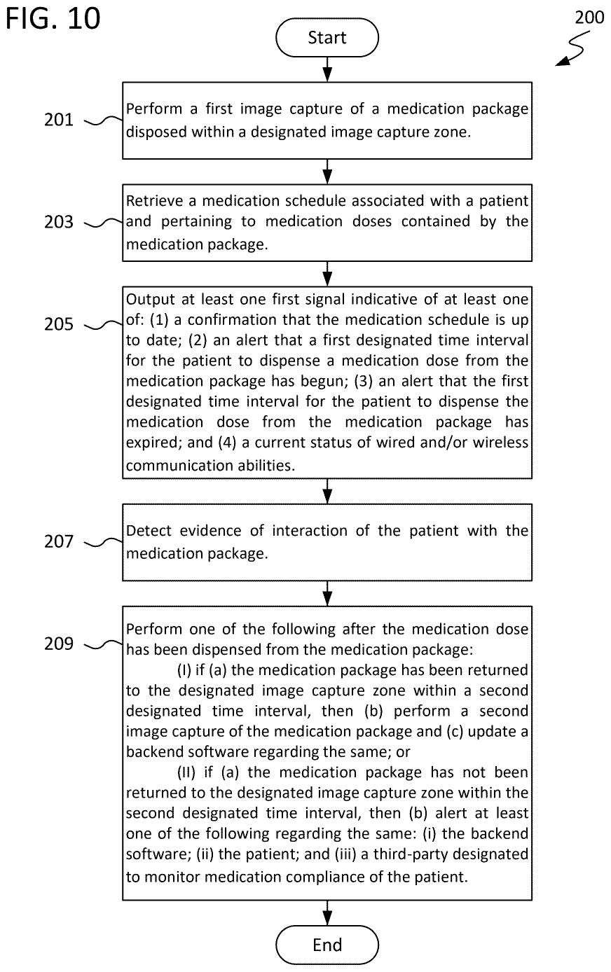

In accordance with some other embodiments, system 100 may be configured to operatively interface with a computing device 20 in performance of any of its various functions described herein. For instance, consider FIGS. 8-9, which illustrate several views of a machine vision-based medication compliance monitoring system 100 incorporating a computing device 20 in accordance with some other embodiments of the present disclosure. As can be seen, system 100 may be configured to host a computing device 20, which may be a mobile computing device or otherwise. Examples of some suitable computing devices 20 may include a mobile phone (e.g., a smartphone), a tablet computer, a laptop or notebook computer, a sub-notebook computer, a desktop computer, a personal digital assistant (PDA), a cellular handset, a television set, a computer monitor, and a video conferencing system, among other possibilities. As will be appreciated, system 100 may be configured to utilize one or more image capture devices 140 native to computing device 20 and/or one or more displays 160 and attendant user interfacing feature(s) (discussed below).

System 100 may be configured for use with any of a wide range of compatible medication package types. For instance, in accordance with some embodiments, system 100 may be compatible with a medication package 10 configured as a blister pack (e.g., a plastic container including a plurality of compartments, often arranged in a matrix of rows and columns, each containing a pharmaceutical or other drug dosage and being sealed with a thin foil layer or other frangible material). Other suitable medication package 10 types for use with system 100 will depend on a given target application or end-use and will be apparent in light of this disclosure.

In accordance with some embodiments, system 100 may include one or more indicators 150, which may include any one (or combination) of optical and audio output devices. For instance, indicator(s) 150 may include an optical output device, which may be a light source, such as a light-emitting diode (LED) or other solid-state light engine, or any other device capable of emitting light of a given wavelength, optionally with a given emission period or pattern. Additionally (or alternatively), indicator(s) 150 may include an audio output device, which may be a speaker, beeper, or any other device capable of emitting sound of a given frequency, optionally with a given emission period or pattern. In accordance with some embodiments, a given indicator 150 may be configured to output audio output signal(s) and/or optical output signal(s) indicative of a given medication access event or other condition or circumstance of interest. Other suitable configurations and uses for indicator(s) 150 including audio and/or optical output devices will depend on a given application and will be apparent in light of this disclosure.

In accordance with some embodiments, system 100 may include a display 160, which may be configured to display images, video, text, or any other desired content, including any elements of a user interface (UI) or graphical user interface (GUI). In some cases, display 160 may be integrated with one or more of base portion 110, image capture device mounting portion 120, and medication package receiving portion 130. In other cases, display 160 may be a stand-alone component configured to communicate with one or more other portions of system 100 using any suitable wired and/or wireless communications techniques.

In accordance with some embodiments, display 160 may be configured to receive input via one or more touch-sensitive elements. For instance, in some embodiments, display 160 optionally may be touch-sensitive, in part or in whole. To that end, display 160 may be configured to utilize any one, or combination, of resistive and capacitive touch-sensing or any other suitable touch-sensing techniques, as will be apparent in light of this disclosure. Display 160 may be configured to detect or otherwise sense direct contact and/or the proximate presence of a user's finger, a stylus, or other suitable touch-sensitive-compatible implement at a given touch-sensitive location of display 160. Display 160 (or system 100 more generally) may be configured to translate such input into an electronic signal that may be processed and manipulated or otherwise used to trigger a given UI/GUI action.

In accordance with some embodiments, display 160 may be utilized in one or more of the following listed in Table #2 below:

TABLE #2

1. Medication/vital sign monitoring reminder(s);
2. Adherence feedback;
3. Encouragement messaging;
4. Assistance request(s);
5. Medication reordering;
6. Symptom consultation(s); and
7. System status check(s).

### Medication Compliance Monitoring Methodology

FIG. 8 is a flow diagram of a method 200 of medication compliance monitoring utilizing a machine vision-based medication compliance monitoring system 100 in accordance with an embodiment of the present disclosure. As can be seen, method 200 may begin, as in block 201, with performing, via system 100, a first image capture of a medication package 10 disposed within a designated image capture zone 145 of system 100.

Method 200 may continue, as in block 203, with retrieving, via system 100, a medication schedule associated with a patient and pertaining to medication doses contained by medication package 10.

Method 200 may continue, as in block 205, with outputting, via system 100, at least one first signal indicative of at least one of: (1) a confirmation that the medication schedule is up to date; (2) an alert that a first designated time interval for the patient to dispense a medication dose from medication package 10 has begun; (3) an alert that the first designated time interval for the patient to dispense the medication dose from medication package 10 has expired; and (4) a current status of wired and/or wireless communication abilities of system 100.

Method 200 may continue, as in block 207, with detecting, via system 100, evidence of interaction of the patient with medication package 10. In accordance with some embodiments, the detecting evidence of interaction of the patient with the medication package 10 may include detecting removal and return of medication package 10 from a field of view of system 100 utilizing at least one of machine vision and proximity sensing. In accordance with some embodiments, in detecting evidence of interaction of the patient with medication package 10, system 100 may be configured to detect and analyze medication interaction both locally and remote to system 100.

Method 200 may continue, as in block 209, with performing, via system 100, one of the following after the medication dose has been dispensed from medication package 10: (I) if (a) medication package 10 has been returned to designated image capture zone 145 within a second designated time interval, then (b) perform, via system 100, a second image capture of medication package 10 and (c) update, via system 100, a backend software regarding the same; or (II) if (a) medication package 10 has not been returned to designated image capture zone 145 within the second designated time interval, then (b) alert, via system 100, at least one of the following regarding the same: (i) the backend software; (ii) the patient; and (iii) a third-party designated to monitor medication compliance of the patient.

As will be appreciated in light of this disclosure, method 200 optionally may be expanded or adapted to include any of a wide range of additional actions of interest. For instance, in accordance with some embodiments, method 200 also may include learning, via system 100, a plurality of forms of medication packages 10. Additionally (or alternatively), method 200 may include applying, via system 100, a date and a timestamp associated with a given target activity, in accordance with some embodiments. Additionally (or alternatively), method 200 may include detecting and recording, via system 100, an undesired or excess dosage activity, in accordance with some embodiments. Other suitable actions of interest for optional inclusion in method 200 will depend on a given target application or end-use and will be apparent in light of this disclosure.

Vital Signs Monitoring and Association with Medication Compliance Monitoring

In accordance with some embodiments, method 200 optionally may be expanded or adapted to include one or more actions pertaining to vital signs monitoring and association thereof with medication compliance. For instance, in some embodiments, method 200 may include: retrieving, via system 100, a schedule for obtaining at least one vital sign measurement associated with the patient; receiving, from a source external to system 100, data pertaining to at least one vital sign measurement associated with the patient; and sending, via system 100, at least a portion of the data pertaining to the at least one vital sign measurement along with time and date information to a database external to system 100. In some embodiments, method 200 further may include outputting, via system 100, at least one second signal indicative of at least one of: the at least one vital sign measurement; and an event in the schedule for obtaining the at least one vital sign measurement. Additionally (or alternatively), in some embodiments, method 200 may include: analyzing, via system 100, a relationship between (a) a medication compliance determination associated with the patient and (b) the at least one vital sign measurement.

### Additional Details and Example Embodiments

In accordance with some embodiments, system 100 generally may be configured as an artificial intelligence (AI)-based vision system that monitors and encourages medication adherence. System 100 may include a defined region (e.g., a medication tray) and an integrated monitoring system (e.g., camera or video camera, computer, LED lights, sensors, local and wide-area communications, custom-developed AI visioning algorithms/software) that observes the defined region for medication activity.

In accordance with some embodiments, utilization of system 100 may proceed, in part or in whole, as in Table #3 below:

TABLE #3
1. A pharmacist may package medication(s) in a unit dose configuration in a blister pack. The
medication schedule then may be uploaded into backend software, and the prepared blister pack
may be sent to the patient.
2. Upon receipt, the patient may place the blister pack on the medication tray and turn on the
monitoring function of system 100. System 100 initially may sense the presence of the blister
pack and download the medication schedule for that patient.
3. If the medication(s) are up to date, indicator 150 may light up (e.g., as a green LED ring).
Indicator 150 may change color (e.g., turn blue) when it is time to take the medication. If the
medication time window is missed, indicator 150 may change color (e.g., turn red), and software
alerts also may be sent by system 100. A sound option is available to complement the visual
indication(s). If there is no Wi-Fi and/or cellular data service, indicator 150 visually may
indicate as such, and system 100 may do a store-and-forward operation until communication is
restored. Additionally, system 100 may check in with the backend software periodically (e.g.,
every six hours) to verify that system 100 is working properly if there is no activity during that
period.
4. To take the medication, the patient may pick up the blister pack, expel the medication dose
from the next available blister cell, take the medication, and place the blister pack back on the
medication tray. System 100 may sense the return of the blister pack, identify the medication
taking event, and update the backend software accordingly. If the blister pack is not returned to
the medication tray within a target time window (e.g., five minutes), indicator 150 may change
color (e.g., turn red), and system 100 may alert the backend software accordingly. The patient
(and/or other designated recipient) may be notified of the patient's medication status through a
series of alerts via digital communication.
5. System 100 also may be trained and deployed for other types of unit dose medication
packaging beyond blister packs. Examples may include a pill box organizer and pill pouch bags,
among others.
6. System 100 may be augmented to receive remote patient vital sign data. If this option is
enabled, system 100 may download the patient's vital sign-taking schedule. When system 100
receives the patient's vital signs, it may date and timestamp them and forward them to a server
database (e.g., which optionally may be cloud-based, in part or in whole) for recording and
analysis. With this option, system 100 also may display a visual status for the vital sign
requirement(s) and incorporate a digital reminder system.
7. The backend software optionally may utilize AI-based software to determine associations
between the medication scorecard and vital sign accumulation at the individual patient and
cross-patient levels.

The foregoing description of example embodiments has been presented for the purposes of illustration and description. It is not intended to be exhaustive or to limit the present disclosure to the precise forms disclosed. Many modifications and variations are possible in light of this disclosure. It is intended that the scope of the present disclosure not be limited by this detailed description. Future-filed applications claiming priority to this application may claim the disclosed subject matter in a different manner and generally may include any set of one or more limitations as variously disclosed or otherwise demonstrated herein.

What is claimed is:

1. A method of medication compliance monitoring utilizing a machine vision-based medication compliance monitoring system, the method comprising:
performing, via the system, a first image capture of a medication package disposed within a designated image capture zone of the system;
retrieving, via the system, a medication schedule associated with a patient and pertaining to medication doses contained by the medication package;
outputting, via the system, at least one first signal indicative of at least one of:
a confirmation that the medication schedule is up to date;
an alert that a first designated time interval for the patient to dispense a medication dose from the medication package has begun;
an alert that the first designated time interval for the patient to dispense the medication dose from the medication package has expired; and
a current status of wired and/or wireless communication abilities of the system; and
detecting, via the system, evidence of interaction of the patient with the medication package;
after the medication dose has been dispensed from the medication package, performing, via the system, one of the following:
(I) if (a) the medication package has been returned to the designated image capture zone within a second designated time interval, then (b) perform, via the system, a second image capture of the medication package and (c) update, via the system, a backend software regarding the same; or
(II) if (a) the medication package has not been returned to the designated image capture zone within the second designated time interval, then (b) alert, via the system, at least one of the following regarding the same:
the backend software;
the patient; and
a third-party designated to monitor medication compliance of the patient.

2. The method of claim 1, wherein the detecting evidence of interaction of the patient with the medication package comprises:
detecting removal and return of the medication package from a field of view of the system utilizing at least one of machine vision and proximity sensing.

3. The method of claim 1, wherein in detecting evidence of interaction of the patient with the medication package, the system is configured to detect and analyze medication interaction both locally and remote to the system.

4. The method of claim 1, further comprising:
learning, via the system, a plurality of forms of medication packages.

5. The method of claim 1, further comprising:
applying, via the system, a date and a timestamp associated with a given target activity.

6. The method of claim 1, further comprising:
detecting and recording, via the system, an undesired or excess dosage activity.

7. The method of claim 1, further comprising:
retrieving, via the system, a schedule for obtaining at least one vital sign measurement associated with the patient;
receiving, from a source external to the system, data pertaining to the at least one vital sign measurement associated with the patient; and
sending, via the system, at least a portion of the data pertaining to the at least one vital sign measurement along with time and date information to a database external to the system.

8. The method of claim 7, further comprising:
outputting, via the system, at least one second signal indicative of at least one of:
the at least one vital sign measurement; and
an event in the schedule for obtaining the at least one vital sign measurement.

9. The method of claim 8, wherein the at least one second signal is indicative of both:
the at least one vital sign measurement; and
an event in the schedule for obtaining the at least one vital sign measurement.

10. The method of claim 7, further comprising:
analyzing, via the system, a relationship between (a) a medication compliance determination associated with the patient and (b) the at least one vital sign measurement.

11. A machine vision-based medication compliance monitoring system configured to perform the method of claim 1.

12. The system of claim 11, wherein the system comprises at least one image capture device configured to perform at least one of:
the first image capture; and
the second image capture.

13. The system of claim 12, wherein the at least one image capture device is a component native to a computing device.

14. The system of claim 13, wherein the computing device is a smartphone or a tablet computer.

15. The system of claim 11, wherein the at least one image capture device is configured with a fixed field of view.

16. The system of claim 11, wherein the at least one image capture device is configured with an adjustable field of view.

17. The system of claim 11, wherein the system comprises at least one indicator configured to output at least one of:
the at least one first signal; and
the at least one second signal.

18. The system of claim 11, wherein the system comprises at least one display configured to provide a user interface or graphical user interface.

19. The system of claim 18, wherein the at least one display is touch-sensitive.

20. The system of claim 18, wherein the at least one display is native to a computing device.
